(19) United States
(12) Patent Application Publication
(10) Pub. No.: US 2012/0004722 A1
(43) Pub. Date: Jan. 5, 2012

(54) DEGRADATION RESISTANT IMPLANTABLE MATERIALS AND METHODS

(75) Inventors: Alexei Goraltchouk, Santa Barbara, CA (US); Jordan M. Thompson, Scotts Valley, CA (US); Miriam M. Abiad, Costa Mesa, CA (US); Kevin A. Ma, Scotts Valley, CA (US)

(73) Assignee: ALLERGAN, INC., Irvine, CA (US)

(21) Appl. No.: 13/104,893

(22) Filed: May 10, 2011

Related U.S. Application Data
(63) Continuation-in-part of application No. 13/015,309, filed on Jan. 27, 2011.
Application No. 13/015,309 is a continuation-in-part of application No. 13/021,615, filed on Feb. 4, 2011.
(60) Provisional application No. 61/301,104, filed on Feb. 3, 2010.
(60) Provisional application No. 61/375,338, filed on Aug. 20, 2010.
(60) Provisional application No. 61/301,864, filed on Feb. 5, 2010.

(51) Int. Cl. A61F 2/12; B05D 1/36; B05D 3/00

(52) U.S. Cl. 623/8; 427/2.24

(57) ABSTRACT

Methods are provided for making materials suitable for implantation in a mammal. The methods include the steps of providing a base material having a desirable surface topography, such as a polyurethane foam, contacting the base member with a silicone-based fluid material to form a coating, and allowing the coating to set to form a silicone-based structure suitable for implantation in a mammal. The base material may be removed from the coating.

### RELATED APPLICATIONS

This application is a continuation in part and claims priority pursuant to 35 U.S.C. §120 to U.S. patent application Ser. No. 13/015,309, filed Jan. 27, 2011, which claims priority benefit to U.S. Provisional Application Ser. No. 61/301,104, filed on Feb. 3, 2010, and claims priority benefit to U.S. Provisional Application Ser. No. 61/375,338, filed Aug. 20, 2010; this application is also a continuation in part and claims priority pursuant to 35 U.S.C. §120 to U.S. patent application Ser. No. 13/021,615, filed Feb. 4, 2011, which claims priority benefit to U.S. Provisional Application Ser. No. 61/301,864, filed on Feb. 5, 2010; each of which is hereby incorporated by reference in its entirety.

The present invention generally relates to medical implants and more specifically relates to foam-like materials suitable for implantation in a mammal.

Prostheses or implants for augmentation and/or reconstruction of the human body are well known. Capsular contracture is a complication associated with surgical implantation of prostheses, particularly with soft implants, and even more particularly, though certainly not exclusively, with fluid-filled breast implants.

Capsular contracture is believed to be a result of the immune system response to the presence of a foreign material in the body. A normal response of the body to the presence of a newly implanted object, for example a breast implant, is to form a capsule of tissue, primarily collagen fibers, around the implant. Capsular contracture occurs when the capsule begins to contract and squeeze the implant. This contracture can be discomforting or even extremely painful, and can cause distortion of the appearance of the augmented or reconstructed breast. The exact cause of contracture is not known. However, some factors may include bacterial contamination of the implant prior to placement, submuscular versus subgladular placement, and smooth surface implants versus textured surface implants, and bleeding or trauma to the area.

Surface texturing has been shown to reduce capsular contracture when compared to what are known as “smooth” surface implants.

There is still a need for a more optimal surface textured implant that further reduces the potential for capsular contracture. The present invention addressed this need.

### SUMMARY OF THE INVENTION

Accordingly, the present invention provides a method of making a material suitable for implantation in a mammal. The method generally comprises the steps of providing a base member including a porous surface defined by interconnected pores and contacting the base member with a silicone-based fluid material in a manner to cause the fluid material to enter the pores. In one embodiment, a vacuum is applied to the base member to draw the fluid material into and/or through the pores. The method may comprise the steps of removing excess fluid material from the base member to obtain a coating of the fluid material on the porous surface, and allowing the coating to set to form a silicone-based structure suitable for implantation in a mammal. The removal process can be obtained using an airknife to blow away the excess material, and/or squeezing out the excess material, and/or using suction to remove the excess material. The silicone-based structure includes a porous surface, having interconnected cells, the porous surface substantially identically conforming to the porous surface of the base member.

In one aspect of the invention, the base material is a material which can be degraded or otherwise removed from within the coating without substantially affecting the coating structure. In some embodiments, the base material is a substantially biodegradable material. The base material may be polyurethane, for example, polyurethane foam. Alternatively, the base member is melamine, for example, melamine foam. Other base member materials are also contemplated and include, for example, foams made from polyethylene, polyethylene vinyl acetate, polystyrene, polyvinyl alcohol, or generally a polyolefin, polyester, polyether, polyamide, polysaccharide, a material which contains aromatic or aliphatic structures in the backbone, as functionalities, crosslinkers or pendant groups, or a copolymer, terpolymer or quarternaly polymer thereof. Alternatively the material may be a composite of one or more aforementioned materials. In another embodiment of the invention the base material can be a metal, for example a metal foam, a ceramic, or a composite material.

The silicone-based fluid material may comprise a dispersion, for example, a silicone dispersion, solution, emulsion or mixture. The silicone-based fluid material may be a solution of a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

In another aspect of the invention, the base member, or at least a portion thereof, is removed from the silicone-based structure. In one embodiment, substantially all of the base material is removed, such that a product is obtained which comprises or consists of material that is substantially entirely pure silicone, for example, a porous, cellular silicone foam. The step of removing may comprise, for example, contacting the base member with a solution capable of dissolving the base member. For example, in an embodiment of the invention in which the base member is polyurethane foam, the step of removing may comprise contacting the base member with a hydrogen peroxide solution. In other embodiments of the invention, the base material may be degraded by exposure to UV light, heat, oxidative agents, a base such as sodium hydroxide, or an acid such as phosphoric acid or a combination thereof. The material may be exhaustively removed further by a secondary process such as solvent leach or vacuum.

In another aspect of the invention, a material suitable for implantation in a mammal is provided. The material comprises a porous, cellular member comprising a silicone-based structure. The silicone-based structure has a topography, for example, a pore size, shape and interconnectivity, substantially identical to that of a polyurethane foam. This material may be made by the processes in accordance with methods of the invention, as described herein.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable polymeric structure suitable for implantation in a mammal. More specifically, the method includes contacting the base member with a fluid precursor of the substantially non-biodegradable polymeric material in a manner to cause the fluid precursor to enter the pores, removing excess fluid precursor material to obtain a coating of the fluid precursor on the base member, and allowing the coating to set to form the substantially non-biodegradable polymeric structure. The resulting structure includes a porous surface substantially identically conforming to the porous surface of the base member.

In yet another aspect of the invention, a method is provided which generally comprises providing a base member including a porous surface defined by interconnected pores, contacting the base member with a first material, allowing the first material to set to form a first material coating on the base member, contacting the first material coating with a second material different from the first material and allowing the second material to set to form a layered polymeric structure suitable for implantation in a mammal. The resulting layered polymeric structure includes a porous surface substantially identically conforming to the porous surface of the base member. In an exemplary embodiment, the first material is a fluorinated polyolefin material and the second material is a silicone dispersion.

In yet further aspects of the invention, methods for augmenting or reconstructing a human breast are provided, wherein the methods comprise implanting, in a human breast, a material made by the methods described herein.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is an SEM micrograph of a implantable material made in accordance with a method of the invention; and.

FIG. 2 is an SEM micrograph of a melamine foam which can be used as a base member in accordance with a method of the invention.

FIGS. 3-9 are images of other materials that can be useful as base materials in accordance with different embodiments of the invention.

FIG. 10 illustrates an exemplary general method scheme.

FIG. 11 A illustrates a cross-sectional SEM image of a base material strut as described herein. FIGS. 11 B-D illustrate SEM images two coatings, four coatings and six coatings of a fluid material respectively on a base material strut.

FIG. 12 A illustrates an SEM image of a base material as described herein. FIGS. 12 B-D illustrate SEM images of two coatings, four coatings and six coatings of a fluid material respectively on a base material.

FIG. 13 A illustrates an SEM image of a foam-like material as described herein with the base material removed. FIGS. 13 B-E illustrate SEM images of one coating, two coatings, three coatings and four coatings of a fluid material respectively on the foam-like material.

FIG. 14 are bar graphs showing data of thickness and disorganization of capsules from various biomaterials, normalized to Textured 1 biomaterial. FIG. 14A shows a bar graph of thickness data as normalized mean±normalized standard deviation. FIG. 14B shows a bar graph of disorganization normalized with a standard deviation with upper and lower bounds of confidence intervals.

FIG. 15 is bar graph showing data of collagen content of capsules formed over various biomaterials (n=6). Results are shown as mean±standard deviation. Asterisks (*) indicates a statistically significant from Texture 1 biomaterial.

FIG. 16 is a bar graph showing data from a tissue adhesion test of various biomaterials. Results are shown as mean±standard deviation.

FIG. 17 is bar graph showing data of stiffness of capsule/ingrowth formed over various tissue expanders at time 0 and at 6 weeks (n=8). Results are shown as mean±standard deviation.

### DETAILED DESCRIPTION

The present invention generally pertains to implantable materials and methods of forming implantable materials. The materials may be used as coverings or outer layers for implants, such as breast implants, and are designed to at least reduce the risk of capsular contracture.

In one aspect of the invention, methods are provided for making an implantable material that is substantially biologically inert and/or substantially non-biodegradable, which has a structure, for example, a microstructure, similar or substantially identical to that of a foam of a different material. The different material may be, or may not be, a biologically inert or non-biodegradable material.

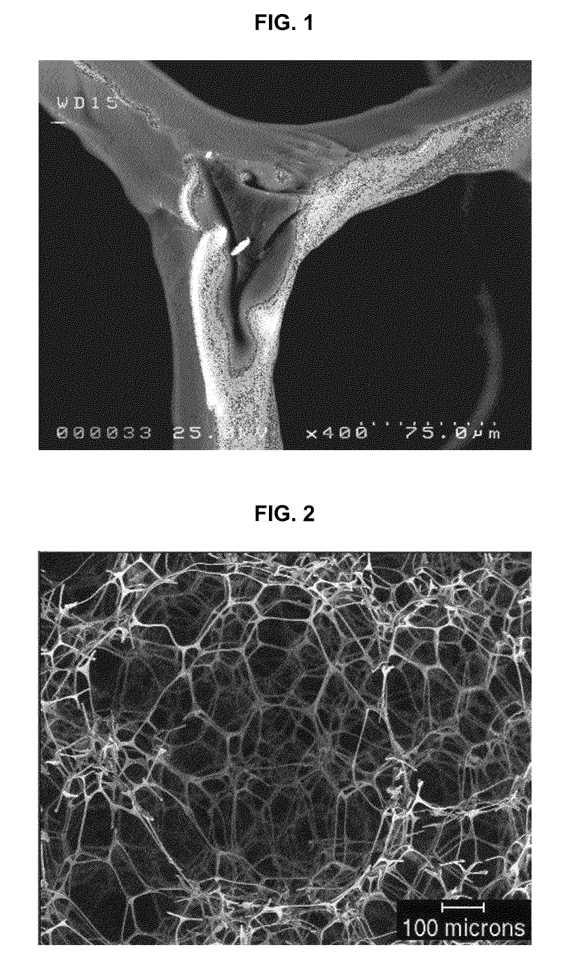

In a specific embodiment, the implantable materials are substantially entirely comprised of silicone yet have the topographical structure of a polyurethane foam. For example, a material in accordance with one embodiment is a flexible, soft, silicone-based foam having substantially the same or substantially identical geometry and tissue disorganization potential of a polyurethane foam, but with the chemical inertness and biocompatibility of a silicone. FIG. 1 is an SEM image of a polyurethane foam strut coated with silicone elastomer, in accordance with an embodiment of the invention.

For example, a method for making an implantable material substantially entirely comprised of silicone, in accordance with one embodiment of the invention, generally comprises the steps of providing a polyurethane base member including a porous surface defined by interconnected pores, contacting the base member with a silicone-based fluid material in a manner to cause the fluid material to enter the pores. A vacuum may be applied to the base material in order to facilitate the contacting step. Excess fluid material may be removed from the base member to obtain a coating of the fluid material on the porous surface. The silicone-based coating is allowed to set to form a silicone-based structure. The coating steps may be repeated once, twice, three or more times, for example, up to 1000 times, until a desired thickness and/or final foam density is achieved. The underlying polyurethane material may be removed from the coating structure. For example, the polyurethane is contacted with a dissolvent, dimethyl sulfoxide, or a degradant such as hydrogen peroxide or hydrochloric acid, followed by a dissolvent such as dimethyl sulfoxide of dimethyl formamide or acetone. The resulting silicone-based material is flexible and biocompatible and includes a porous surface substantially identically conforming to the porous surface of a polyurethane foam.

It is to be appreciated that for a base material other than polyurethane, said base material can be removed by a solvent or other means, known to those of skill in the art, suitable for removing the base material from the coating without substantially altering or affecting the coating structure.

The base material may have a pore size of about 100-1000 μm (RSD, i.e. relative standard deviation, of about 0.01-100%); an interconnection size of about 30-700 μm (RSD of 0.01-100%); interconnections per pore of about 2-20(RSD of 0.01-50%); and an average pore to interconnection size ratio of about 3-99%.

In some embodiments, the base material has a pore size of about 300-700 μm (RSD of 1-40%); an interconnection size of about 100-300 μm (RSD of 1-40%); interconnections per pore of about 3-10 (RSD of 1-25%) and an average pore to interconnection size ratio of about 10-99%.

In an exemplary embodiment, the base member comprises a material, for example, polyurethane or other suitable material, having a pore size of 472+/−61 μm (RSD=13%), interconnection size: 206+/−60 μm (RSD=29%), interconnections per pore: 9.6+/−1.8 (RSD=19%), Pore to interconnection size ratio of 44%.

The base member may comprise any suitable porous material having the desired surface structure. Alternative to polyurethane, the base member may comprise melamine, for example, melamine foam. FIG. 2 is an SEM micrograph of a melamine foam having a topography defined by highly interconnected, open pores. Other base member materials useful in the methods of the invention are also contemplated and include, for example, polyethylene foam, Styrofoam, or general polyolefin foams, polysaccharide foams, polyamide foams, polyacrylate foams, metal and ceramic foams.

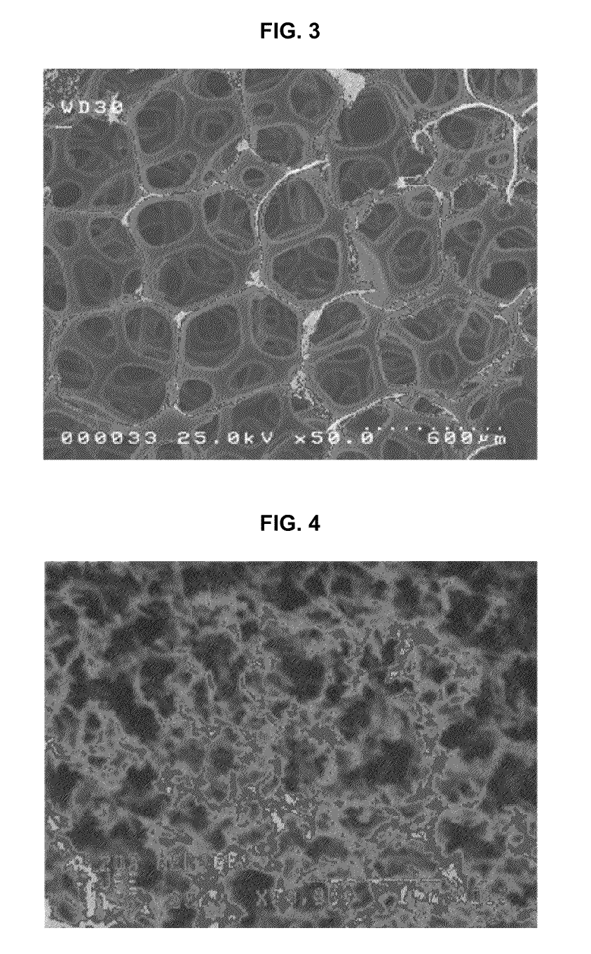

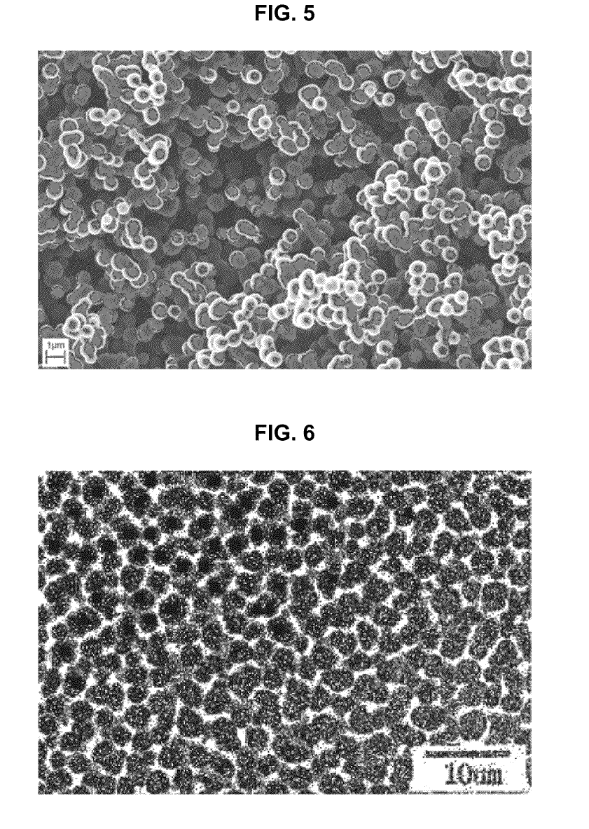

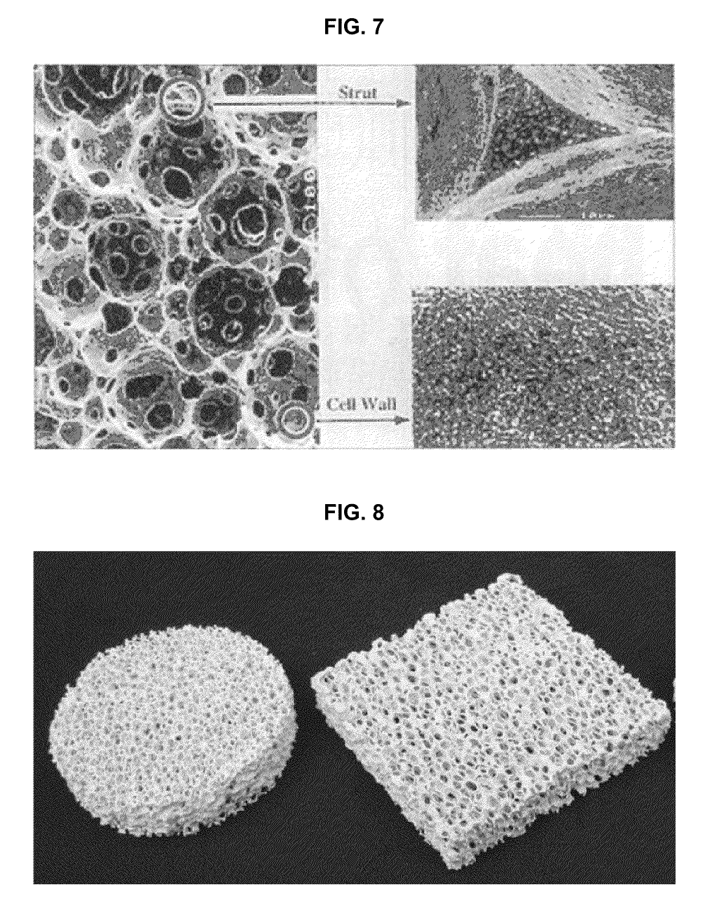

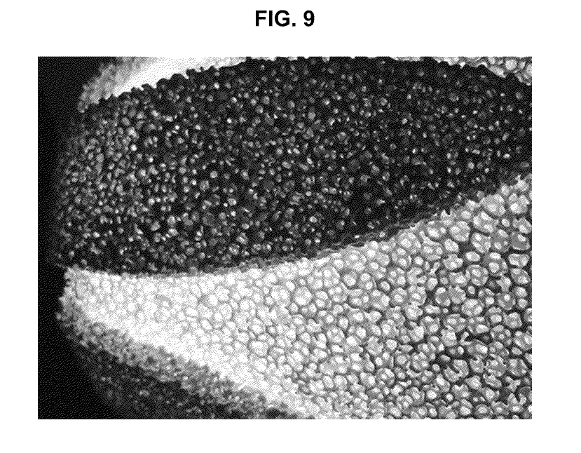

Porous surfaces of base member materials useful in accordance with various embodiments of the invention are shown in FIGS. 3-10. More specifically, FIG. 3 is a SEM image of a polyurethane foam base; FIG. 4 is an alumina aerogel foam; FIG. 5 is another aerogel, for example, silica aerogel foam; FIG. 6 is a silica foam; FIG. 7 is a HiP foam; FIG. 8 is a magnesium ceramic foam; and FIG. 9 is another ceramic foam.

In an exemplary embodiment, the silicone-based fluid material may comprise a dispersion, for example, a silicone dispersion. The silicone-based fluid material may be a room temperature vulcanizing (RTV) or a high temperature vulcanizing (HTV) silicone. In an exemplary embodiment, the silicone-based fluid material is a high temperature vulcanizing (HTV) platinum-cured silicone dispersion in xylene or chloroform.

Alternatives to silicone-based polymers are also contemplated. For example, any implantable material that can be cured by crosslinking, thermoplastics that set by change in temperature, material that set by removal of solvents or any elastomer that cures or sets by any known mechanism, can be used. It is further contemplated that other implantable materials useful in accordance with the invention include suitable metals or ceramics.

The type of polymeric fluid material forming the coating on the base member, the total dissolved solids of the coating material, the method of removing the excess fluid, the carrier solvent, the method of applying the coating solution, the temperature of the solution, can be varied in accordance with different embodiments of the invention.

In some embodiments, base material is coated with multiple layers of different materials. For example, a first coating material may comprise a barrier layer of a material capable of reducing or preventing diffusion of chemical substances from the base material, and a second coating applied on the first coating may comprise a silicone-based material. Other coating materials may be selected to achieve various characteristics of the final product, such as materials to strengthen the foam, prevent chemical degradation, and/or change surface properties.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable composite structure suitable for implantation in a mammal. For example, the base member may comprise a polyurethane foam. The substantially non-biodegradable polymeric material can be any suitable biocompatible polymer and may be selected from a list of highly impermeable systems such as fluorinated polymers to prevent diffusion of chemical entities which may facilitate the degradation of polyurethane. Alternatively, the fluorinated polymer can be applied as a base layer, prior to a final application of the silicone, to act as a barrier layer.

For example, in one embodiment of the invention, a method of making a textured material, for example, but not limited a porous material suitable for implantation in a mammal, is provided wherein the method comprises the steps of providing a base material comprising polyurethane foam having a surface defined by interconnected pores and contacting the base material with a fluorinated polymeric material in a manner to cause the fluorinated polymeric material to enter the pores. A vacuum and/or air blower or airknife may be applied as described elsewhere herein to facilitate intimate and uniform contact between the materials. The composite material thus formed has a fluorinated polymer surface defined by interconnected pores that are substantially identical to those of the polyurethane foam surface. In one embodiment, the fluorinated polymeric material is a fluorinated polyolefin. In another embodiment, the method may further comprise the step of contacting the fluorinated polymeric surface with a silicone-based material in a manner to form a silicone-based coating on the fluorinated polymeric surface. A textured prosthesis may be assembled by applying or attaching this composite material to a surface of an implantable device, for example, a breast prosthesis.

In another embodiment of this invention, the base member of a preferred geometry, that is not dissolvable (for example, a crosslinked polymer having a porous surface) may be coated by a robust but dissolvable material, such as, for example, a foam material selected from the group of materials consisting of polystyrene, polyethylene-co-vinyl acetate, and poly(styrene-co-butadiene-co-styrene). The base member, e.g. the non-dissolvable foam, can then be removed from the dissolvable material coating, for example, degraded by relatively aggressive means, for example, by acid digestion in 37% HCl, leaving the robust but dissolvable material behind. An implantable material of interest, for example, a silicone-based fluid material, is deposited on the robust but dissolvable foam, for example, using the methods described elsewhere herein. The silicone-based fluid material may be in the form of a dispersion having a solvent system that does not dissolve the robust polymer. The silicone is allowed to set or cure, and the robust material is then dissolved out by means which does not affect the material of interest (e.g. silicone), for example, by dissolution in acetone in the case of polystyrene. In this case, the material of interest is not subjected to aggressive conditions used to dissolve the original foam.

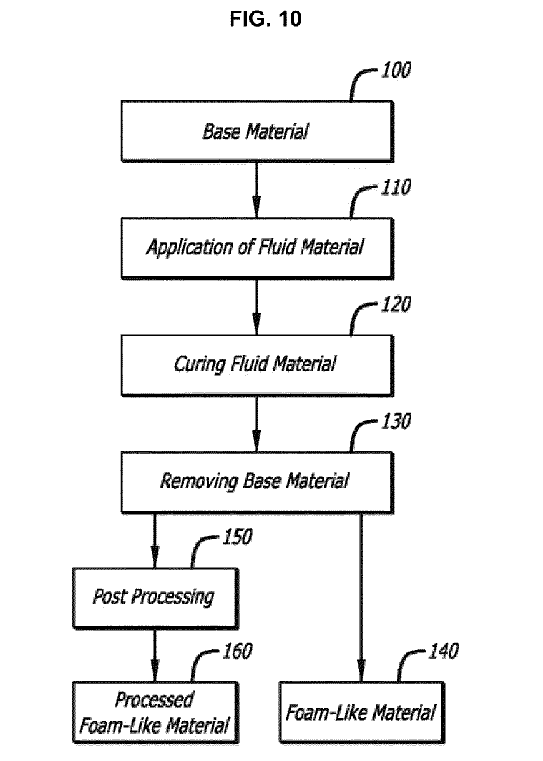

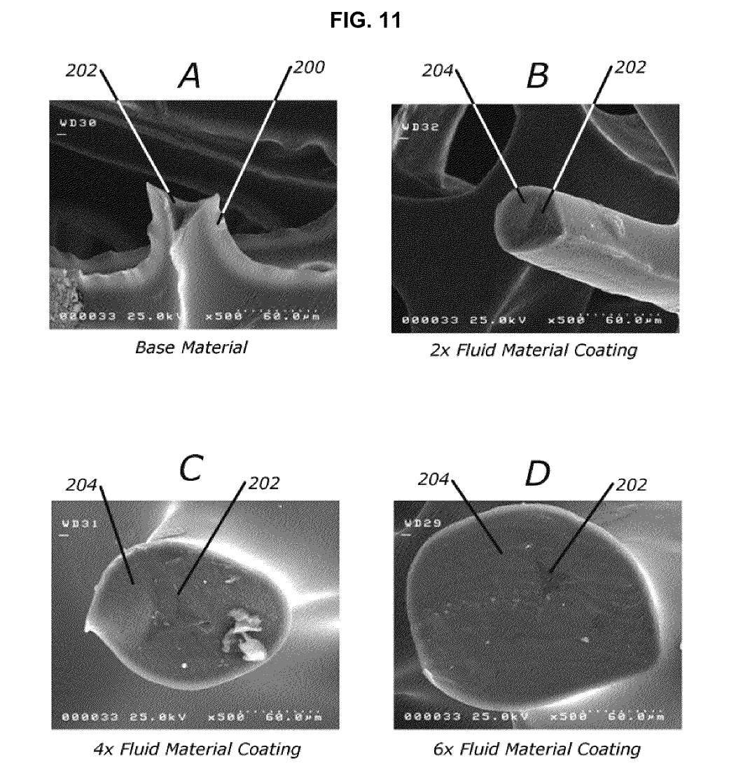

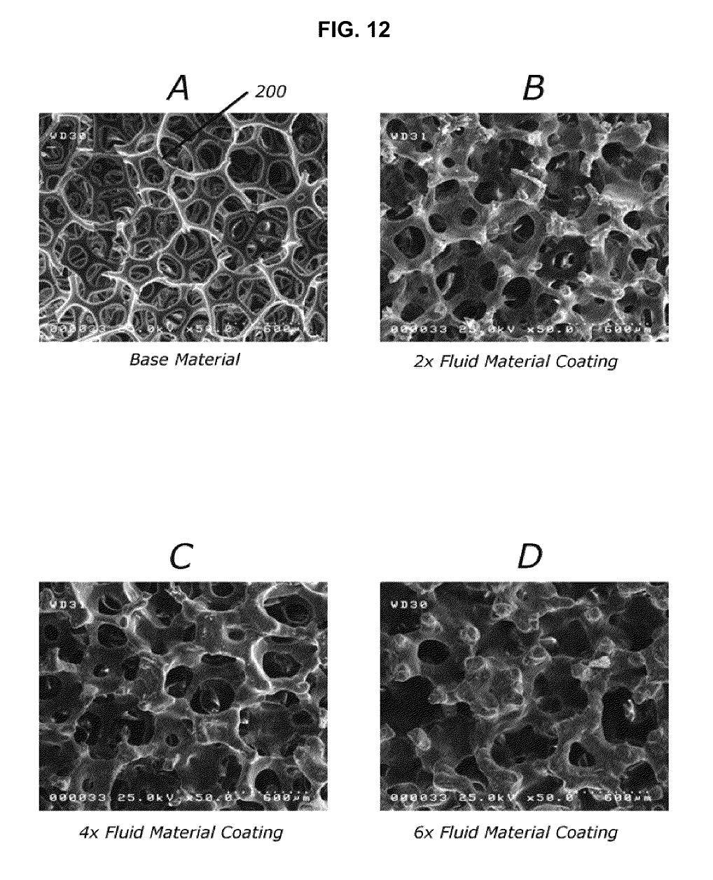

Other methods for producing foam-like materials are described. An overview of exemplary possesses is illustrated in FIG. 10. As a first step 100, an appropriate base material is chosen. FIGS. 11 and 12 illustrate an exemplary base material ready for coating as described herein. As illustrated, the base material forms interconnections between struts 200. FIG. 11A illustrates a cross-section of strut 200 which has a triangular shape 202. It is appreciated by one skilled in the art that a triangular strut shape is one of an almost infinite set of geometries that a strut can assume.

Batch processing, reel to reel processing, and/or conveyor belt processing can be used in the application of one or more fluid materials in order to achieve a high throughput of material, such as on an industrial scale. In a conveyer belt system, formation of a foam-like material or processed foam-like material can be accomplished sequentially in stations or as a continuous process. Bath processing can also be combined with a conveyer belt system wherein several foam-like materials can be produced simultaneously.

In a second step 110, a fluid material is applied to the base material. A fluid material is applied via a coating technique such as, but not limited to, curtain application, spraying, knifing, dipping, and the like. The application of the fluid material can have varying parameters. For example, an airknife blade can be used to remove residual fluid material; however, an airknife need not be use in some embodiments. Likewise, heating of the fluid material can be varied or even not used. Further, a vacuum need or need not be used to facilitate fluid material intrusion into the pores of the base material. Other non-limiting parameters that can be varied include temperature programs, air velocity, pressure, speed of material traveling on conveyor belt, number of coating stations/nozzles, number of airknife stations/nozzles, and number of suction locations to obtain various thicknesses and uniformities of the conformal coat.

Other steps to remove excess fluid material include, but are not limited to, using a vacuum to draw the fluid into the porous surface, using an airknife to blow away excess fluid material, using another means of positive pressure, pressing the base material to squeeze out excess fluid material or a combination of those procedures.

After the fluid material has been properly applied to the base material, the fluid material is cured 120. The fluid material is cured via exposure to an element which activates crosslinking, curing, setting, gelling, solidification, and/or any sort of phase change into a stable form of the fluid material. The method of curing can be different depending on the particular application. For example, RTV silicones can be cured by application of heat, or moist hot air or through addition (e.g., by spraying overtop) of cross-linker and activation of the cross-linker. Hydrogels, on the other hand, can be cross-linked using UV activated cross-linkers, peroxide cross-linkers which are activated by heat, or other cross-linkers which are activated by the addition of a catalyst. Further still, curing can be achieved by simple devolitilization on the conveyer belt, precipitation out of solution, and/or solidification by cooling (e.g., if the polymer is applied in a molten state).

FIGS. 11 B-D illustrate a cross section of strut 200 coated two times (FIG. 11 B), four times (FIG. 11 C) and six times (FIG. 11 D). Triangular strut shape 202 can be seen in each circumstance with the coating layer 204 growing larger and larger with each additional coating step. FIGS. 12 B-D illustrate, again, the porous base material coated two times, four times and six times respectively.

Next, the base material is removed 130, or leached away, leaving a foam like-material 140 of interest. Here the leaching agent can be sprayed and/or curtain coated onto the cured fluid material/base material composite member, and/or the composite member can be passed through a pool of the leaching agent, or through rollers which apply the leaching agent and squeeze out the air. The leaching can be followed in a similar fashion with a washing step to remove the leaching agent and/or help remove the excess unremoved, unwanted material.

Optionally, the coating and curing steps can be repeated using a post processing step 150. The advantage of repeating the coating and curing steps after the leaching is threefold. 1) If the cured fluid material is partly adversely affected during the leaching step, the application of additional fluid material post leaching can help increase the strength of the cured fluid material. 2) If a fluid material of choice is adversely affected by the post leaching step, a primary sacrificial layer of a first fluid material that is not affected by the leaching step is applied first, then the base material is leached out and the fluid material of choice is then applied unto the empty primary sacrificial layer. The primary sacrificial layer can then be leached by an alternative method that would not affect the fluid material of choice. Hence, the fluid material is cured and left behind unaffected. 3) To fill the void created by leaching out the base material.

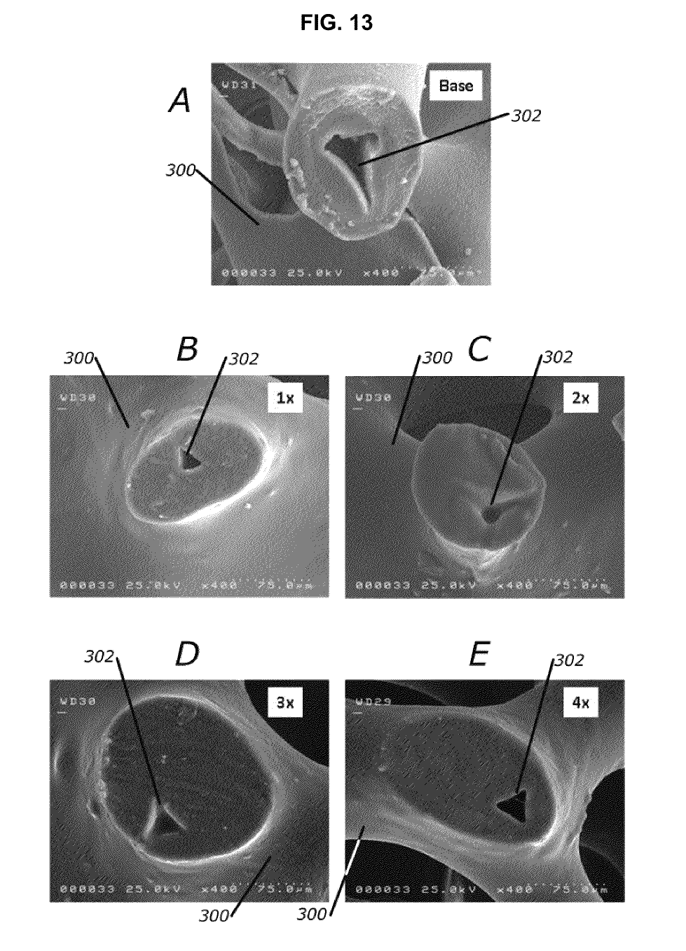

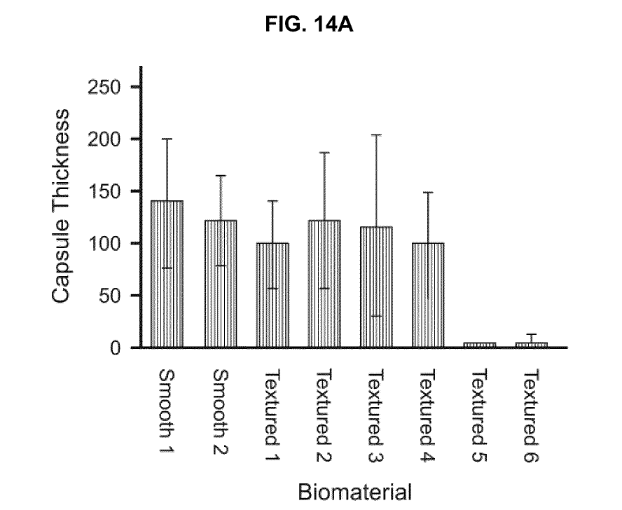

FIG. 13 A illustrates a foam-like material 300 wherein the base material has been leached out. In FIG. 13 A, void 302 exists where a triangular base member strut previously existed. FIGS. 13 B-E illustrate foam-like material 300 further coated one time, two times, three times and four times respectively. FIGS. 13 A-E illustrate the thickness of the material as further coatings are applied to the foam-like material 300.

After the post processing step, a processed foam-like material remains having additional coatings and potentially filled voids wherein the base material previously resided. Such a material can be stronger than a non-processed foam-like material. However, a strong foam-like material can produced in some embodiments without the need for optional post processing step(s). For example, a foam-like material substantially formed from a metal fluid material may not need to be subjected to post processing steps.

The present specification also discloses a method of implanting a prosthesis, the method comprising the step of implanting the prosthesis in a patient, the prosthesis covered by a porous material disclosed herein; wherein at any time after implantation, if a capsule has formed, the capsule has a thickness of 75 μm or less, has fiber disorganization comprising 50% or more of the fibers that are not parallel to the prosthesis surface, has tissue growth into the biomaterial of the prosthesis of 100 μm or more, has less than 40% collagen content, adheres to tissue with a peak force of at least 8 N and/or and has a stiffness of 20 mmHg/mL or less.

The present specification also discloses a method of implanting a prosthesis, the method comprising the step of implanting the prosthesis in a patient, the prosthesis covered by a porous material disclosed herein; wherein at any time after implantation, if a capsule has formed, the capsule has a thickness of 50 μm or less, has fiber disorganization comprising 60% or more of the fibers that are parallel to the prosthesis surface, has tissue growth into the biomaterial of the prosthesis of 125 μm or more, has less than 30% collagen content, adheres to tissue with a peak force of at least 9 N and/or and has a stiffness of 15 mmHg/mL or less.

The present specification also discloses a method of implanting a prosthesis, the method comprising the step of implanting the prosthesis in a patient, the prosthesis covered by a porous material disclosed herein; wherein at any time after implantation, if a capsule has formed, the capsule has a thickness of 25 μm or less, has fiber disorganization comprising 70% or more of the fibers that are not parallel to the prosthesis surface, has tissue growth into the biomaterial of the prosthesis of 150 μm or more, has less than 20% collagen content, adheres to tissue with a peak force of at least 10 N and/or and has a stiffness of 10 mmHg/mL or less.

The present specification also discloses a method of implanting a prosthesis, the method comprising the step of implanting the prosthesis in a patient, the prosthesis covered by a porous material disclosed herein; wherein at any time after implantation, if a capsule has formed, the capsule has a thickness of about 5 μm to about 75 μm, has fiber disorganization comprising about 50% to about 90% of the fibers that are not parallel to the prosthesis surface, has tissue growth into the biomaterial of the prosthesis of about 100 μm to about 300 μm, has about 5% to about 40% collagen content, adheres to tissue with a peak force of about 8 N to about 11 N, and/or and has a stiffness of about 5 mmHg/mL to about 20 mmHg/mL.

### Example 1

A polyurethane open celled foam is coated according to the current invention using a solution of Silicone HTV 30% w/v, by either dipping the polyurethane foam in the solution, casting the solution on a sheet of polyurethane or spraying the solution in excess over the sheet of polyurethane. The excess solution is removed by squeezing out the foam, or by vacuum which is applied through a Buchner funnel at the bottom of the foam (in the case of casting the solution over the foam) or by blowing air over the foam as in the case of an air-knife, or in combination of any of the aforementioned. The foam is then devolitilized in vacuum or by application of mild heat in the case of HTV, such that the solvent is removed, but the HTV is not cured. This can be achieved in the application of the air current during the previous step (the air may or may not be heated). Finally the coated foam is cured and the coating layer is affixed unto the foam. The process may be repeated from 1 to about 1000 times (more specifically 1, 4 times) to achieve various builds (final pore densities). The polyurethane is completely removed from the center of the structure by digestion in hydrogen peroxide/water solution with or without the presence of metal ions and with or without heating. Alternatively the polyurethane foam can be degraded out by 37% HCl digestion for 1-5 minutes, with vigorous agitation and air removal to facilitate the uniform digestion of the polyurethane, and a subsequent DMSO wash to remove the remnant degradants which are not soluble in the 37% HCl. The degradation/leaching steps can be repeated 1-20 times to achieve various levels of purity. The resulting material is a substantially pure silicone foam useful as a surgical implant.

### Example 2

A sheet polyurethane open celled foam (20×20 cm) is placed in a container the bottom of which is a fine grate. Vacuum is applied to the bottom of the grate to pull air through the top of the foam into the foam and finally through the grate and out. A solution of about 20% HTV (platinum cured) in chloroform is cast over the foam and pulled through the foam by the vacuum, a jet of air is applied to the foam through an air-knife to remove any remaining solution droplets that are trapped in the foam to clean out the pores. The foam is then devolitized in vacuum at about room temperature for 2 hours. The devolitized foam is finally cured at 120° C. for 1 hour. The process is repeated 3 times. The resulting foam is an open celled polyurethane base foam, conformally coated by an approximately 50 μm layer of silicone.

### Example 3

A implantable material is produced substantially in accordance with Example 1, except that instead of a polyurethane foam, a melamine foam is used as the base member. In addition, the base material is not removed from the silicone foam. The resulting implantable material comprises a highly porous, open celled structure having a melamine base and a silicone overcoat.

### Example 4

The silicone foam of Example 1 is produced as a flexible sheet. The sheet is cut and laminated to form a front surface of a breast implant. The front surface of the breast implant has a surface texture substantially identical to a surface texture of a polyurethane foam, but is substantially pure silicone.

### Example 5

A sheet of polyurethane open celled foam base material (20×20 cm) is placed in a container the bottom of which is a fine grate. Vacuum is applied to the bottom of the grate to pull air through the top of the foam into the foam and finally through the grate and out. A solution of MED-4850, a high durometer silicone, is cast over the foam and pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a second coating is applied by casting a solution of MED-4830, a lower durometer silicone, over the cured first coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a third coating is applied by casting a solution of MED-4815, an even lower durometer silicone, over the cured second coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

Then, a fourth final coating is applied by casting a solution of MED-4801, the lowest durometer silicone used, over the cured third coating. The solution is pulled through the foam by the vacuum, a jet of air is applied to the foam through an air-knife to remove any remaining solution droplets that are trapped in the foam to clean out the pores. The foam is then devolitized in vacuum at about room temperature for 2 hours and cured at 120° C. for 1 hour.

The resulting material is an open celled polyurethane base foam, conformably coated by an approximately 200 μm layer of decreasing durometer silicone. The polyurethane base material can be optionally removed from the composite member. Other composite materials can be similarly made.

### Example 6

A sheet of polyurethane open celled foam base material (20×20 cm) is placed in a container the bottom of which is a fine grate. Vacuum is applied to the bottom of the grate to pull air through the top of the foam into the foam and finally through the grate and out. An aqueous dispersion of fluorinated polyolefin (e.g. HYPOD™ Polyolefin Dispersions available from DOW Chemical Company) is cast over the foam and pulled through the foam by the vacuum. A jet of air is applied to the foam through an air-knife to remove any remaining solution droplets that are trapped in the foam and to clean out the pores. The fluorinated polyolefin coated foam is then heated at a sufficient temperature to allow the water in the aqueous dispersion to evaporate and the coating to melt. The fluorinated polyolefin coating is a uniform, fine film coating on the surfaces of the polyurethane foam. This coated polyurethane foam can then be bonded with a suitable, biocompatible adhesive to a smooth shell breast prosthesis which can then be implanted in a patient. The prosthesis will have the desirable characteristics of a polyurethane covered implant, that is, for example, the capsular tissue disorganization potential of polyurethane foam, but with the reduced chance of degradation of the polyurethane foam into the body.

### Example 7

A fluorinated polyolefin-coated polyurethane foam material is made as described in Example 6. However, before the material is bonded to a smooth shell breast prosthesis, a silicone coating is applied to the fluorinate polyolefin coating by casting a solution of MED-4830 over the fluorinate polyolefin coating. The silicone solution is pulled through the foam by the vacuum, and a jet of air is applied to the foam through an air-knife to remove any remaining solution droplets that are trapped in the foam to clean out the pores. The foam is then devolitized in vacuum at about room temperature for 2 hours and cured at 120° C. for 1 hour. The coated polyurethane foam is then bonded with a suitable, biocompatible adhesive to a smooth shell breast prosthesis.

### Example 8

### Capsule Thickness and Disorganization

In order to measure the thickness and disorganization of capsules formed, disks (1 cm in diameter) of various porous biomaterials were implanted subcutaneously in Sprague-Dawley rats using standard procedures. The biomaterials tested were taken from commercially available implants or experimentally produced as follows: Smooth 1, a biomaterial having a smooth surface (NATRELLE®, Allergan, Inc., Irvine, Calif.); Smooth 2, a biomaterial having a smooth surface (MEMORYGEL®, Mentor, Inc., Santa Barbara, Calif.); Textured 1, a biomaterial having a closed-cell textured surface produced from a lost-salt method (BIOCELL®, Allergan, Inc., Irvine, Calif.); Textured 2, a biomaterial having a closed-cell textured surface produced from an imprinting method (SILTEX®, Mentor, Inc., Santa Barbara, Calif.); Textured 3, a biomaterial having a closed-cell textured surface produced from either an imprinting or gas foam method (SILIMED®, Sientra, Inc., Santa Barbara, Calif.); Textured 4, a biomaterial having a closed-cell textured surface produced from an imprinting method (Perouse Plastie, Mentor, Inc., Santa Barbara, Calif.); Textured 5, a biomaterial having an open-cell polyurethane surface; Textured 6, a biomaterial having an open-cell textured surface produced according to the methods disclosed herein. Samples were harvested at 6 weeks, fixed in formalin, and processed to produce paraffin blocks. The paraffin blocks were sectioned using a microtome at 2 μm thickness and stained with hematoxylin and eosin (H&E).

Implanted porous biomaterials were characterized by measuring the thickness and disorganization of the capsule formed over the biomaterial. Capsule thickness was measured by acquiring 2 representative 20× images of the H&E stained biomaterials and measuring the thickness of the capsule at 3 points in the image. Capsule disorganization was evaluated by acquiring 3 representative 20× images of the H&E stained biomaterials, and then drawing a reference vector tangent to the implant surface, as well as, drawing vectors along collagen fibers within the capsule. The angle of each vector relative to the reference vector was then measured, and the standard deviation of the angles was calculated, where greater standard deviations reflected a higher degree of disorganization. All image analysis calculations were performed on the Nikon Elements Advanced Research software.

All thickness and disorganization measurements were acquired blinded and each measurement was normalized to the data obtained from Textured 1 biomaterial. For the thickness data collected, a one-way ANOVA was run to determine significant effects (p<0.05). If there were any statistically significant effects from the ANOVA analysis, the Tukey's post-hoc test was run for multiple comparisons at α=0.05. For the disorganization data collected, a Levene's Test for Equal Variance was used to determine whether there was a statistically significant difference in disorganization between experimental groups (p<0.05). Between individual groups, the criteria for non-significance were overlap of confidence intervals (95%), adjusted for the number of groups.

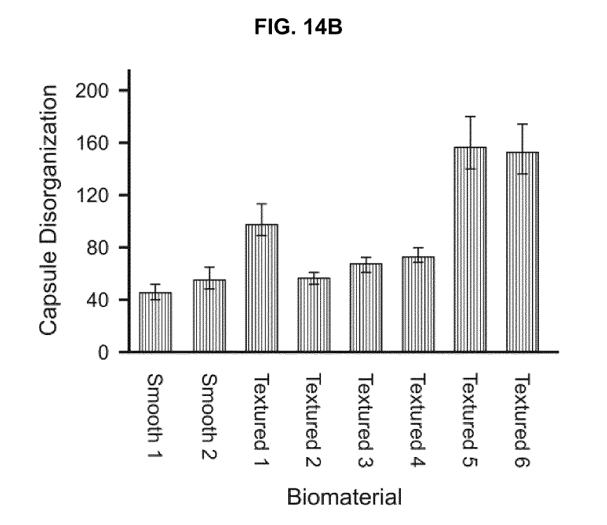

The capsule thicknesses and disorganization, normalized to the Texture 1 biomaterial within each respective study, are shown in FIG. 14. Smooth Texture 1 and 2 biomaterials, and Textures 1-4 biomaterials (having closed-cell texture) exhibited pronounced capsule formation, and the capsules formed were of equivalent thicknesses of about 100 μm to about 140 μm (FIG. 14A). Texture 5-6 biomaterials exhibited minimal capsule formation with capsules formed having a thickness of less than 10 μm (FIG. 14A). With respect to capsule organization, it was found that Texture 1 biomaterial resulted in a capsule that was more disorganized than Smooth 1 and 2 and Texture 2-4 biomaterials (FIG. 14B). Texture 5 and 6 biomaterials demonstrated extensive ingrowth (about 200 μm) that was interconnected and significantly more disorganized (≧50% of fibers were not parallel to implant surface) than Smooth 1 and 2 and Texture 1-4 biomaterials (FIG. 14B). These findings show that Smooth 1 and 2 biomaterials (smooth surface) and Textures 1-4 biomaterials (closed-cell textured surfaces) resulted in a capsule with predominantly organized collagen. Textures 5-6 biomaterials (open-cell textured surfaces), in contrast, induce significant ingrowth that can eliminate capsule and disorganize the tissue at the material-tissue interface.

### Example 7

### Capsule Collagen

In order to measure the collagen content of capsules formed, disks (1 cm in diameter) of various porous biomaterials were implanted subcutaneously in Sprague-Dawley rats using standard procedures. The biomaterials tested were taken from commercially available implants or experimentally produced as follows: Smooth 1, a biomaterial having a smooth surface (NATRELLE®, Allergan, Inc., Irvine, Calif.); Smooth 2, a biomaterial having a smooth surface (MEMORYGEL®, Mentor, Inc., Santa Barbara, Calif.); Textured 1, a biomaterial having a closed-cell textured surface produced from a lost-salt method (BIOCELL®, Allergan, Inc., Irvine, Calif.); Textured 2, a biomaterial having a closed-cell textured surface produced from an imprinting method (SILTEX®, Mentor, Inc., Santa Barbara, Calif.); Textured 3, a biomaterial having a closed-cell textured surface produced from an imprinting method (Perouse Plastie, Mentor, Inc., Santa Barbara, Calif.); Textured 4, a biomaterial having a closed-cell textured surface produced from either an imprinting or gas foam method (SILIMED®, Sientra, Inc., Santa Barbara, Calif.); Textured 5, a biomaterial having an inverse foam polyurethane-polyethylene glycol surface; Textured 6, a biomaterial having an inverse foam polyurethane-polyethylene glycol surface; Textured 7, a biomaterial having an open-cell polyurethane surface; Textured 8, a biomaterial having a non-woven felt surface. Samples were harvested at 6 weeks, fixed in formalin, and processed to produce paraffin blocks. The paraffin blocks were sectioned using a microtome at 2 μm thickness and stained with aniline blue.

Implanted porous biomaterials were characterized by measuring staining darkness of the capsule formed over the biomaterial. The darkness of the capsule was measured from 5 representative 20× images, with overall intensity averaged over the capsules to reflect the depth of staining. To account for variations in parameters, such as section thickness and precise staining times, all measurements were normalized to the intensity measured within the dermis of the same section, which was utilized as a standard due to the consistent staining that was observed in this region. A one-way ANOVA was run to determine significant effects (p<0.05). If there were any statistically significant effects from the ANOVA analysis, the Tukey's post-hoc test was run for multiple comparisons at α=0.05.

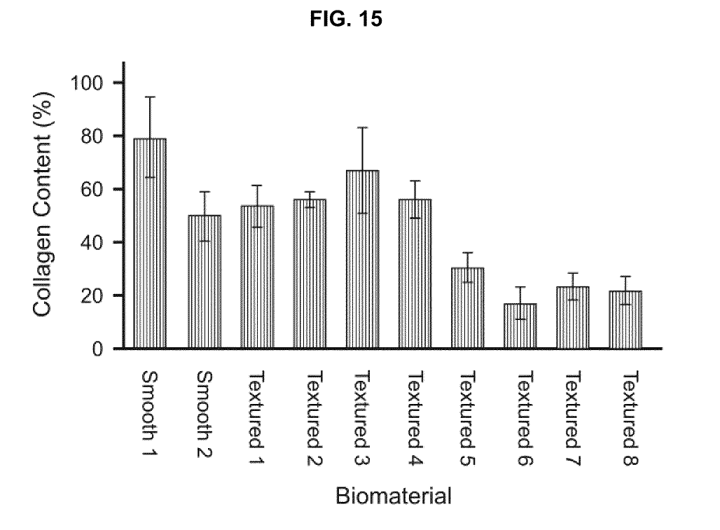

FIG. 15 shows the mean collagen density of capsules and ingrowth formed over smooth and textured biomaterials. It was found that the capsules formed over Smooth 1 and 2 biomaterials and Textured 1-4 biomaterials (closed-cell textured surfaces) showed a statistically significant increase in collagen density over the Texture 5 and 6 biomaterials (inverse foam textured surface), Textured 7 biomaterial (open-cell textured surface), and Textured 8 biomaterial (non-woven felt textured surface). As such, the prevention of capsule formation was shown to be linked to significant ingrowth into an open, interconnected texture, where the ingrowth has a low collagen density.

### Example 8

### Tissue Adhesion

In order to evaluate the effect of texture on tissue adhesion to a porous biomaterial, strips of various biomaterial were implanted subcutaneously in a Sprague-Dawley rat using standard procedures. The biomaterials tested were taken from commercially available implants or experimentally produced as follows: Smooth 1, n=38, a biomaterial having a smooth surface (NATRELLE®, Allergan, Inc., Irvine, Calif.); Textured 1, n=64, a biomaterial having a closed-cell textured surface produced from a lost-salt method (BIOCELL®, Allergan, Inc., Irvine, Calif.); Textured 2, n=6, a biomaterial having a closed-cell textured surface produced from an imprinting method (SILTEX®, Mentor, Inc., Santa Barbara, Calif.); Textured 3, n=6, a biomaterial having an inverse foam polyurethane-polyethylene glycol surface; Textured 4, n=45, a biomaterial having an inverse foam polyurethane-polyethylene glycol surface; Textured 5, n=45, a biomaterial having an open-cell polyurethane surface; Textured 6, n=6, a biomaterial having an open-cell polyurethane surface; Textured 7, n=6, a biomaterial having an open-cell textured surface comprising a methyl silicone elastomer produced according to the methods disclosed herein; Textured 8, n=6, a biomaterial having an open-cell textured surface comprising a fluoropolymer elastomer produced according to the methods disclosed herein. Samples were harvested at 4 weeks, and tissue was pulled from the test strip on a mechanical tester with a pullout speed of 2 mm/second. Adhesion strength was measured as the peak force required to separate the implant from the surrounding tissue. A one-way ANOVA was run to determine significant effects (p<0.05). If there were any statistically significant effects from the ANOVA analysis, the Tukey's post-hoc test was run for multiple comparisons at α=0.05.

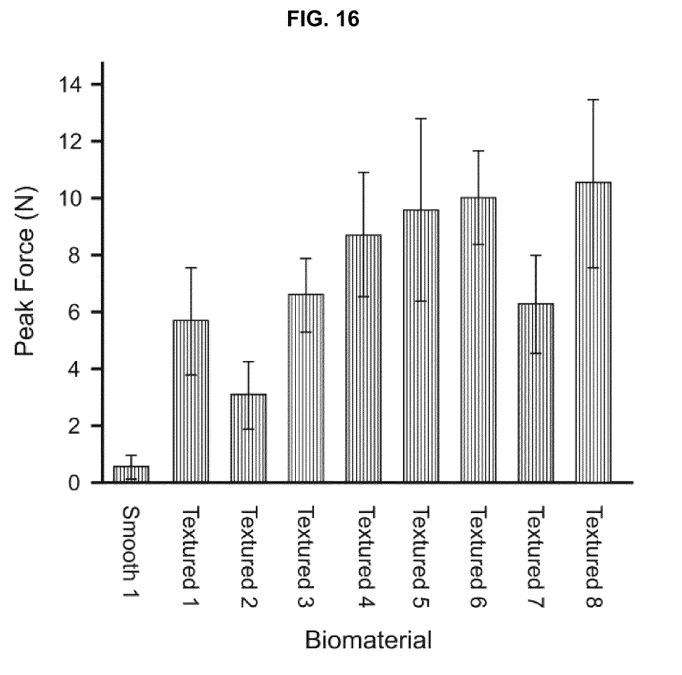

Smooth 1 biomaterial showed little adhesion, as there were no significant protrusions above a micro-scale and had minimal drag on the surrounding tissue (FIG. 16). Textured 1 and 2 biomaterials (closed-cell textured surfaces) exhibited limited amount of tissue interaction and showed greater adhesion than Smooth 1 (FIG. 16). Textured 3 and 4 biomaterials (inverse Foam textured surface) and Textures 5-8 biomaterials (open-cell textured surfaces) showed the highest degree of tissue adhesion (FIG. 16). As such, Textured 5-8 biomaterials promoted significant tissue infiltration/ingrowth because of the highly porous and interconnected textures.

### Example 9

### Capsule Stiffness

In order to evaluate stiffness of capsules/ingrowth formed over a porous biomaterial, 7 mL mini-expanders comprising silicone biomaterial of various textures were implanted subcutaneously in a Sprague-Dawley rat using standard procedures. The biomaterials tested were taken from commercially available implants or experimentally produced as follows: Smooth 1, a biomaterial having a smooth surface (NATRELLE®, Allergan, Inc., Irvine, Calif.); Textured 1, a biomaterial having a closed-cell textured surface produced from a lost-salt method (BIOCELL®, Allergan, Inc., Irvine, Calif.); Textured 2, a biomaterial having an open-cell textured surface produced according to the methods disclosed herein. At time 0 (immediately post-implantation) and at 6 weeks, saline was incrementally added to each expander, and the resulting pressure exerted on and by the expander at each step was measured with a digital manometer. Stiffness was calculated by fitting a trend-line to the linear region of the pressure-volume curve and measuring the slope of the line. Increases in the stiffness of the capsule/ingrowth were reflected by increases in the slope. To account for expander-to-expander variability, each stiffness measurement was normalized to the stiffness of the expander itself. A one-way ANOVA was run to determine significant effects (p<0.05). If there were any statistically significant effects from the ANOVA analysis, the Tukey's post-hoc test was run for multiple comparisons at α=0.05.

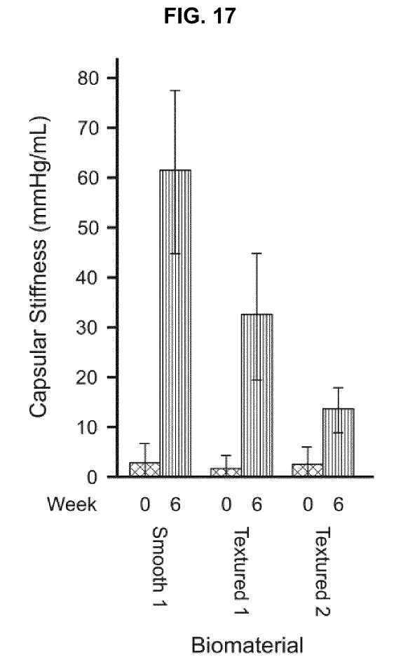

Capsules formed over Smooth 1 biomaterial expander showed the greatest stiffness after 6 weeks (FIG. 17). Textured 1 biomaterial expander (closed-cell textured surface) showed lower stiffness than Smooth 1 biomaterial expander but greater stiffness than the Textured 2 and 3 biomaterial expanders (open-cell textured surface) (FIG. 17). This data demonstrates that closed-cell biomaterials result in capsules that are stiffer than those that result from open-cell biomaterials that support ingrowth and prevent capsule formation.

### Example 10

### Bleeding and Capsule Response

In order to identify critical morphological and physical characteristics of the porous biomaterials disclosed herein, disks (1 cm in diameter) of various biomaterials were implanted subcutaneously in a Sprague-Dawley rat using standard procedures and the response to such implantation in terms of capsule formation and bleeding were determined. The morphological and physical characteristics tested for each biomaterial are given in Tables 1 and 2.

TABLE 1
Morphological Characteristics of Biomaterials

| | | | | | Mean |
|---|---|---|---|---|---|
| | Mean | Mean | Mean pore | Mean | interconnection |
| | thickness | porosity | size | interconnections/ | size |
| Biomaterial | (mm) | (%) | (μm) | pore | (μm) |
| Polyurethane | 2.40 ± 0.10 | 98.0 ± 0.4 | 522 ± 87 | 14.2 ± 3.2 | 166 ± 48 |
| Polyurethane | 2.90 ± 0.01 | 98.0 ± 0.0 | 488 ± 119 | 14.2 ± 1.4 | 230 ± 69 |
| Mesh | 0.89 ± 0.06 | 72.2 ± 1.5 | 522 ± 137 | N/A | N/A |
| Mesh | 1.38 ± 0.10 | 70.9 ± 2.9 | 560 ± 134 | N/A | N/A |
| Templated 1 | 1.93 ± 0.52 | 74.0 ± 6.7 | 761 ± 163 | 8.6 ± 3.0 | 271 ± 51 |
| Templated 2 | 2.10 ± 0.1 | 81.8 ± 0.6 | 641 ± 45 | 7.0 ± 3.1 | 200 ± 62 |
| Templated 3 | 2.22 ± 0.14 | 85.8 ± 0.7 | 551 ± 60 | 8.3 ± 3.0 | 161 ± 49 |
| Templated 4 | 2.30 ± 0.10 | 77.1 ± 1.6 | 538 ± 76 | 7.4 ± 2.0 | 157 ± 40 |
| Templated 5 | 2.46 ± 0.20 | 80.5 ± 2.3 | 874 ± 211 | 6.9 ± 2.4 | 292 ± 69 |
| Templated 6 | 2.58 ± 0.11 | 81.8 ± 0.9 | 683 ± 143 | 7.0 ± 2.2 | 235 ± 98 |
| Templated 7 | 2.58 ± 0.09 | 80.5 ± 1.1 | 676 ± 128 | 6.1 ± 1.6 | 264 ± 109 |
| Templated 8 | 2.69 ± 0.04 | 73.4 ± 1.5 | 473 ± 80 | 6.7 ± 0.8 | 237 ± 70 |
| Templated 9 | 2.72 ± 0.04 | 78.0 ± 1.4 | 548 ± 113 | 7.8 ± 1.5 | 203 ± 65 |
| Templated | 2.73 ± 0.02 | 65.3 ± 1.0 | 511 ± 108 | 6.7 ± 1.5 | 217 ± 62 |
| 10 | | | | | |
| Templated | 2.73 ± 0.04 | 83.1 ± 1.6 | 573 ± 90 | 8.3 ± 2.8 | 172 ± 58 |
| 11 | | | | | |
| Templated | 2.77 ± 0.05 | 52.4 ± 2.9 | 455 ± 90 | 6.0 ± 1.1 | 172 ± 53 |
| 12 | | | | | |
| Templated | 2.78 ± 0.03 | 57.6 ± 1.5 | 457 ± 73 | 5.6 ± 0.7 | 199 ± 67 |
| 13 | | | | | |
| Templated | 2.79 ± 0.00 | 81.3 ± 0.1 | 579 ± 61 | 8.6 ± 2.5 | 142 ± 48 |
| 14 | | | | | |
| Templated | 2.80 ± 0.00 | 79.1 ± 1.0 | 578 ± 73 | 7.3 ± 1.7 | 159 ± 59 |
| 15 | | | | | |
| Templated | 2.83 ± 0.03 | 84.5 ± 0.3 | 634 ± 57 | 8.6 ± 2.7 | 175 ± 56 |
| 16 | | | | | |
| Templated | 2.83 ± 0.01 | 78.2 ± 0.7 | 487 ± 106 | 9.3 ± 2.1 | 160 ± 51 |
| 17 | | | | | |
| Templated | 2.86 ± 0.02 | 77.9 ± 1.3 | 489 ± 91 | 7.5 ± 1.3 | 149 ± 46 |
| 18 | | | | | |
| Templated | 2.87 ± 0.01 | 80.2 ± 1.4 | 497 ± 94 | 9.2 ± 2.0 | 145 ± 34 |
| 19 | | | | | |
| Templated | 2.87 ± 0.07 | 75.0 ± 2.0 | 430 ± 93 | 7.8 ± 2.1 | 145 ± 38 |
| 20 | | | | | |
| Templated | 2.88 ± 0.04 | 79.3 ± 1.3 | 468 ± 87 | 8.5 ± 2.3 | 161 ± 50 |
| 21 | | | | | |
| Templated | 2.89 ± 0.02 | 77.2 ± 0.6 | 445 ± 94 | 8.7 ± 2.5 | 159 ± 48 |
| 22 | | | | | |
| Templated | 2.90 ± 0.00 | 66.7 ± 2.1 | 624 ± 38 | 7.9 ± 1.8 | 177 ± 47 |
| 23 | | | | | |
| Templated | 2.90 ± 0.00 | 78.1 ± 1.5 | 478 ± 96 | 8.61 ± 2.24 | 150 ± 43 |
| 24 | | | | | |
| Templated | 2.91 ± 0.01 | 77.2 ± 1.9 | 513 ± 86 | 8.0 ± 1.5 | 141 ± 46 |
| 25 | | | | | |
| Templated | 2.91 ± 0.12 | 79.5 ± 2.4 | 506 ± 109 | 8.9 ± 3.1 | 139 ± 35 |
| 26 | | | | | |
| Templated | 2.93 ± 0.03 | 76.9 ± 0.9 | 571 ± 73 | 7.6 ± 2.8 | 159 ± 45 |
| 27 | | | | | |
| Templated | 2.93 ± 0.02 | 76.1 ± 1.6 | 426 ± 52 | 7.2 ± 1.6 | 161 ± 51 |
| 28 | | | | | |
| Templated | 2.94 ± 0.10 | 70.0 ± 2.5 | 493 ± 66 | 8.7 ± 1.9 | 145 ± 46 |
| 29 | | | | | |
| Templated | 2.95 ± 0.05 | 71.1 ± 1.8 | 493 ± 89 | 8.1 ± 2.2 | 172 ± 34 |
| 30 | | | | | |
| Templated | 2.98 ± 0.09 | 69.7 ± 1.2 | 498 ± 66 | 10.0 ± 0.8 | 174 ± 41 |
| 31 | | | | | |

TABLE 2
Physical Characteristics of Biomaterials

| | Compressive Response | | | |
|---|---|---|---|---|
| | at 5% | at 10% | at 20% | Elongation |
| Biomaterial | strain | strain | strain | at Break |
| Polyurethane | 1.74 ± 0.40 | 2.60 ± 0.53 | 3.38 ± 0.52 | N/D |
| Polyurethane | 1.41 ± 0.13 | 2.63 ± 0.03 | 2.89 ± 0.20 | 454 ± 7 |
| Mesh | 0.07 ± 0.01 | 0.20 ± 0.02 | 0.74 ± 0.08 | 336 ± 39 |
| Mesh | 0.09 ± 0.04 | 0.22 ± 0.09 | 0.74 ± 0.39 | 439 ± 56 |
| Templated 1 | 0.08 ± 0.09 | 0.24 ± 0.28 | 0.63 ± 0.66 | 327 ± 16 |
| Templated 2 | 0.26 ± 0.10 | 0.60 ± 0.20 | 1.34 ± 0.36 | N/D |
| Templated 3 | 0.29 ± 0.11 | 0.52 ± 0.13 | 1.11 ± 0.23 | N/D |
| Templated 4 | 0.62 ± 0.10 | 2.41 ± 0.33 | N/D | 528 ± 151 |
| Templated 5 | 0.23 ± 0.21 | 0.73 ± 0.64 | 1.64 ± 0.99 | 140 ± 9 |
| Templated 6 | 0.55 ± 0.13 | 1.82 ± 0.75 | 3.87 ± 1.30 | 268 |
| Templated 7 | 0.36 ± 0.20 | 1.26 ± 0.85 | 3.14 ± 1.93 | 213 |
| Templated 8 | 0.84 ± 0.20 | 2.34 ± 0.54 | 5.81 ± 1.07 | 837 ± 73 |
| Templated 9 | 0.77 ± 0.21 | 1.87 ± 0.47 | 3.89 ± 0.87 | 609 ± 62 |
| Templated | 2.70 ± 0.35 | 6.04 ± 0.69 | 12.71 ± 0.99 | 1058 ± 64 |
| 10 | | | | |
| Templated | 0.66 ± 0.13 | 1.15 ± 0.05 | 1.91 ± 0.09 | N/D |
| 11 | | | | |
| Templated | 2.07 ± 0.65 | 4.95 ± 1.61 | 14.18 ± 1.21 | 1065 ± 37 |
| 12 | | | | |
| Templated | 3.80 ± 0.12 | 8.98 ± 0.34 | 19.43 ± 0.37 | 1092 ± 24 |
| 13 | | | | |
| Templated | 1.59 ± 0.19 | 3.05 ± 0.13 | 5.06 ± 0.14 | N/D |
| 14 | | | | |
| Templated | 1.57 ± 0.07 | 3.92 ± 0.46 | 6.06 ± 0.97 | N/D |
| 15 | | | | |
| Templated | 1.12 ± 0.41 | 2.05 ± 0.24 | 3.05 ± 0.26 | N/D |
| 16 | | | | |
| Templated | 0.56 ± 0.06 | 2.11 ± 0.08 | 4.55 ± 0.08 | 658 ± 19 |
| 17 | | | | |
| Templated | 0.43 ± 0.12 | 1.79 ± 0.36 | 4.08 ± 0.79 | 626 ± 157 |
| 18 | | | | |
| Templated | 0.32 ± 0.05 | 1.18 ± 0.19 | 2.88 ± 0.57 | 467 ± 194 |
| 19 | | | | |
| Templated | 0.52 ± 0.19 | 2.29 ± 0.36 | N/D | 674 ± 182 |
| 20 | | | | |
| Templated | 0.48 ± 0.06 | 1.78 ± 0.15 | 3.92 ± 0.41 | 626 ± 170 |
| 21 | | | | |
| Templated | 0.58 ± 0.08 | 2.24 ± 0.20 | N/D | 655 ± 26 |
| 22 | | | | |
| Templated | 2.57 ± 0.73 | 7.97 ± 1.15 | 16.36 ± 2.59 | N/D |
| 23 | | | | |
| Templated | 0.46 ± 0.09 | 1.81 ± 0.24 | 3.75 ± 0.45 | 605 ± 133 |
| 24 | | | | |
| Templated | 0.44 ± 0.08 | 1.86 ± 0.45 | 4.19 ± 0.40 | 683 ± 187 |
| 25 | | | | |
| Templated | 0.34 ± 0.08 | 1.24 ± 0.24 | 3.19 ± 0.76 | 471 ± 152 |
| 26 | | | | |
| Templated | 1.72 ± 0.29 | 4.17 ± 0.66 | 7.63 ± 1.38 | N/D |
| 27 | | | | |
| Templated | 0.58 ± 0.03 | 2.22 ± 0.23 | N/D | 700 ± 127 |
| 28 | | | | |
| Templated | 0.17 ± 0.17 | 1.18 ± 0.72 | 4.30 | 885 ± 14 |
| 29 | | | | |
| Templated | 0.37 ± 0.16 | 1.76 ± 0.35 | N/D | 741 ± 13 |
| 30 | | | | |
| Templated | 0.48 ± 0.14 | 2.24 ± 0.39 | N/D | 663 ± 18 |
| 31 | | | | |

Implanted porous biomaterials were harvested, fixed in formalin, and processed to produce paraffin blocks. The paraffin blocks were sectioned using a microtome at 2 μm thickness and stained with hematoxylin and eosin (H&E). Depending on the morphological characteristic being assessed, capsule response was measured by acquiring at least 3 representative 1×, 4×, 20×, or 50× images of sectioned biomaterial, digitally capturing the images, and measuring the characteristic at 3 or more point in each captured image. All image analysis calculations were performed on the Nikon Elements Advanced Research software. Bleeding response and physical characteristics were measured using routine methods. See, e.g., Winnie, Softness Measurements for Open-Cell Foam Materials and Human Soft Tissue, Measurement Science and Technology (2006).

The summary of the results obtained from this analysis are given in Table 3. The results indicate that porous biomaterials having a wide range in porosity are well tolerated in that in only a very narrow range of porosity (74-86%) was bleeding observed in some of the biomaterials tested. In terms of capsule formation, increased porosity resulted in decreased capsule formation. Interconnection diameter between pores also influenced the bleeding response in that increased diameter resulted in a decreased bleeding response (Table 3). More strikingly, increasing the number of interconnections per pore decreased both the bleeding response and capsule formation seen in the animals in response to the implanted porous biomaterials (Table 3). Lastly, a fine balance in the stiffness of a biomaterial, as measured by compressive forces, was needed to provide the optimal in vivo responses. This is because increased stiffness of a biomaterial resulted in decreased bleeding, whereas decreased stiffness was needed in order to decease capsule formation (Table 3).

TABLE 3
Bleeding and Capsule Response of Biomaterials

| | Bleeding Response | | Capsule Response | |
|---|---|---|---|---|
| Characteristic | No Bleeding | Bleeding | No Capsule | Capsule |
| Mean thickness | 0.6-2.9 | 1.6-2.3 | 0.8-2.9 | 0.6-2.9 |
| (mm) | | | | |
| Mean porosity (%) | 56-98 | 74-86 | 72-98 | 56-81 |
| Mean pore size (μm) | 408-624 | 456-608 | 456-641 | 408-634 |
| Mean | 7-14 | 4.9-9.5 | 7.1-14 | 4.6-9.5 |
| interconnections/ | | | | |
| pore | | | | |
| Mean | 118-325 | 130-153 | 456-641 | 408-634 |
| interconnection | | | | |
| size (μm) | | | | |
| Compressive at 5% | 0.05-2.57 | 0.10-0.20 | 0.05-1.70 | 0.00-2.57 |
| strain (kPa) | | | | |
| Compressive at 10% | 0.18-8.00 | 0.10-4.20 | 0.22-4.17 | 0.10-8.00 |
| strain (kPa) | | | | |
| Compressive at 20% | 1.00-16.0 | 1.90-3.50 | 1.10-7.60 | 0.90-16.0 |
| strain (kPa) | | | | |

Analyzing all the data obtained from these experiments revealed optimal morphological and physical characteristics for a porous material produced from the templating method disclosed herein, was as follows: having a porosity of about 75% to about 83%, having an interconnection size of about 140 μm to about 170 μm, having about 6 to about 10 interconnections per pore, having a compressive force of about 0.60 kPa to about 0.80 kPa at 5% strain, having a compressive force of about 1.7 kPa to about 2.5 kPa at 10% strain, and having a compressive force of about 3.0 kPa to about 5.0 kPa at 20% strain. In an aspect of this embodiment, optimal morphological and physical characteristics for a porous material produced from the templating method disclosed herein, was as follows: having a porosity of about 77% to about 81%, having an interconnection size of about 150 μm to about 160 μm, having about 7 to about 9 interconnections per pore, having a compressive force of about 0.65 kPa to about 0.75 kPa at 5% strain, having a compressive force of about 2.0 kPa to about 2.4 kPa at 10% strain, and having a compressive force of about 3.5 kPa to about 4.5 kPa at 20% strain.

While this invention has been described with respect to various specific examples and embodiments, it is to be understood that the invention is not limited thereto and that it can be variously practiced within the scope of the invention.

What is claimed is:

1. A method of making a material suitable for implantation in a mammal, the method comprising:
providing a base member including a porous surface defined by interconnected pores;
contacting the base member with a silicone-based fluid material in a manner to cause the fluid material to enter the pores;
removing excess fluid material from the base member to obtain a coating of the fluid material on the porous surface; and
allowing the coating to set to form a silicone-based structure suitable for implantation in a mammal, the silicone-based structure including a porous surface substantially identically conforming to the porous surface of the base member.

2. The method of claim 1 further comprising the step of applying a vacuum to the base member to draw the fluid material into the pores.

3. The method of claim 1 further comprising removing at least a portion of the base member from the silicone-based structure.

4. The method of claim 1 wherein the base member is substantially polyurethane.

5. The method of claim 1 wherein the base member is substantially melamine.

6. The method of claim 1 wherein the coating has a thickness of between about 10 microns and about 100 microns.

7. The method of claim 1 further comprising the step of removing substantially all of the base member from the coating after the step of allowing the coating to set.

8. The method of claim 7 wherein the step of removing comprises contacting the base member with a solution capable of dissolving the base member.

9. A method of making a material suitable for implantation in a mammal, the method comprising:
providing a base member comprising a biodegradable foam and including a porous surface defined by interconnected pores;
contacting the base member with a fluid precursor of a substantially non-biodegradable polymeric material in a manner to cause the fluid precursor to enter the pores;
removing excess fluid precursor from the base member to obtain a coating of the fluid precursor on the porous surface; and
allowing the coating to set to form a substantially non-biodegradable polymeric structure suitable for implantation in a mammal, the substantially non-biodegradable polymeric structure including a porous surface substantially identically conforming to the porous surface of the base member.

10. The method of claim 9 wherein the biodegradable foam is a polyurethane foam.

11. The method of claim 9 further comprising the step of removing at least a portion of the base member from the substantially non-biodegradable polymeric structure.

12. The method of claim 9 wherein the substantially non-biodegradable polymeric structure is substantially entirely silicone.

13. A method of making a material suitable for implantation in a mammal, the method comprising:
providing a base member made of a biodegradable foam and including a porous surface defined by interconnected pores;
contacting the base member with a fluorinated polyolefin material in a manner to cause the fluorinated polyolefin material to enter the pores;
allowing the fluorinated polyolefin material to set to form a fluorinated polyolefin coating on the base member;
contacting the fluorinated polyolefin coating with a silicone-based fluid material;
allowing the silicone-based fluid material to set to form a layered polymeric structure suitable for implantation in a mammal, the layered polymeric structure including a porous surface substantially identically conforming to the porous surface of the base member.

14. A method of making a material suitable for implantation in a mammal, the method comprising:
providing a polymeric base member having a surface defined by a geometry including interconnected pores;
forming a first coating on the surface of the base member material, the first coating being selected from the group of materials consisting of polystyrene, polyethylene-co-vinyl acetate, and poly(styrene-co-butadiene-co-styrene);
removing the polymeric base member by contacting the base material with a material that will cause the base member to be removed from the first coating without causing degradation of the coating;
applying a silicone-based fluid material to the first coating having the polymeric base member removed therefrom;
curing the silicone-based fluid material to form a silicone-based coating on the first coating; and
removing the first coating from the silicone coating.

15. The method of claim 14 wherein the step of removing the first coating comprises contacting the first coating with a material that dissolves the first coating without substantially affecting the silicone coating.

16. A method of making a material suitable for implantation in a mammal, the method comprising:
providing a base material comprising polyurethane foam having a surface defined by interconnected pores;
contacting the base material with a fluorinated polymeric material in a manner to cause the fluorinated polymeric material to enter the pores and form a fluorinated polymeric coating on the base material thereby forming a biocompatible, substantially non-biodegradable composite material.

17. The method of claim 16 wherein the fluorinated polymeric material is a fluorinated polyolefin.

18. The method of claim 16 further comprising the step of applying a silicone-based material to the fluorinated polymeric coating in a manner to form a conformal silicone-based coating on the fluorinated polymeric coating.

19. The method of claim 16 further comprising the step of applying vacuum to the base material during the step of contacting.

20. A composite material made by the method of claim 16.

21. A method for augmenting or reconstructing a human breast comprising the steps of:
implanting the material made by the method of claim 1 in a human breast.
